(19) United States
(12) Patent Application Publication
(10) Pub. No.: US 2019/0361437 A1
(43) Pub. Date: Nov. 28, 2019

(54) AUTONOMOUS VEHICLE COORDINATION WITH MEDICAL FACILITY

(71) Applicant: International Business Machines Corporation, Armonk, NY (US)

(72) Inventors: John D. Wilson, League City, TX (US); Kelley Anders, East New Market, MD (US); Jeremy R. Fox, Georgetown, TX (US)

(21) Appl. No.: 15/985,863

(22) Filed: May 22, 2018

(51) Int. Cl. G05D 1/00; G01C 21/34; G16H 80/00; G16H 10/60; H04W 4/90; H04W 4/40; A61B 5/00; G06F 17/28

(52) U.S. Cl. CPC G05D 1/0088; G01C 21/3407; G16H 80/00; G16H 10/60; H04W 4/90; G06F 17/28; A61B 5/747; A61B 5/746; A61B 5/7282; A61B 5/6893; H04W 4/40

(57) ABSTRACT

A medical system in an autonomous vehicle detects a medical condition associated with a passenger in the autonomous vehicle. The medical system identifies a first medical facility and sends the first medical facility a first data package including a location of the autonomous vehicle, the medical condition, passenger information, and a list of actions executable by the autonomous vehicle. The autonomous vehicle receives a second data package from the first medical facility including an authorized action selected from the list of actions. The autonomous vehicle executes the authorized action.

### BACKGROUND

The present disclosure relates to automated medical assistance, and, more specifically, to interactions between an autonomous vehicle and a medical facility during a medical emergency.

### SUMMARY

Aspects of the present disclosure are directed toward a computer-implemented method for managing an autonomous vehicle during a medical emergency, the method can comprise detecting, based on sensor data received from a plurality of sensors communicatively coupled to the autonomous vehicle, a medical condition associated with a passenger in the autonomous vehicle. The method can further comprise identifying, in response to detecting the medical condition and based on information in a medical facility directory stored in a computer-readable storage medium in the autonomous vehicle, a first medical facility from a set of medical facilities based on a location of the autonomous vehicle, a location of the first medical facility, the medical condition, a set of medical capabilities associated with the first medical facility, and an availability of the first medical facility. The method can further comprise sending, in response to identifying the first medical facility, a first data package to the first medical facility using a cellular network. The first data package can comprise the medical condition, passenger information, the location of the autonomous vehicle, and a list of actions executable by the autonomous vehicle. The method can further comprise receiving, from the cellular network and in response to sending the first data package, a second data package from the first medical facility authorizing a first action selected from the list of actions. The method can further comprise executing the first action in response to receiving the second data package. The first action can comprise a physical modification to the autonomous vehicle.

Further aspects of the present disclosure are directed toward a system comprising a processor and a computer-readable storage medium storing a medical facility directory and program instructions for emergency service functionality which, when executed by the processor, are configured to cause the processor to perform a method. The method can comprise detecting, based on sensor data received from a plurality of sensors communicatively coupled to the autonomous vehicle, a medical condition associated with a passenger in an autonomous vehicle. The method can further comprise identifying, in response to detecting the medical condition and based on information in the medical facility directory, a first medical facility from a set of medical facilities based on a location of the autonomous vehicle, a location of the first medical facility, the medical condition, a set of medical capabilities associated with the first medical facility, and an availability of the first medical facility. The method can further comprise sending, in response to identifying the first medical facility, a first data package to the first medical facility using a cellular network. The first data package can comprise the medical condition, passenger information, the location of the autonomous vehicle, and a list of actions executable by the autonomous vehicle. The method can further comprise receiving, from the cellular network and in response to sending the first data package, a second data package from the first medical facility authorizing a first action selected from the list of actions. The method can further comprise executing the first action in response to receiving the second data package. The first action can comprise a physical modification to the autonomous vehicle.

Further aspects of the present disclosure are directed toward a computer program product comprising a computer readable storage medium having program instructions executable by a processor to cause the processor to perform a method comprising detecting, based on sensor data received from a plurality of sensors communicatively coupled to the autonomous vehicle, a medical condition associated with a passenger in the autonomous vehicle. The method can further comprise identifying, in response to detecting the medical condition and based on information in a medical facility directory stored in the computer readable storage medium, a first medical facility from a set of medical facilities based on a location of the autonomous vehicle, a location of the first medical facility, the medical condition, a set of medical capabilities associated with the first medical facility, and an availability of the first medical facility. The method can further comprise sending, in response to identifying the first medical facility, a first data package to the first medical facility using a cellular network. The first data package can comprise the medical condition, passenger information, the location of the autonomous vehicle, and a list of actions executable by the autonomous vehicle. The method can further comprise receiving, from the cellular network and in response to sending the first data package, a second data package from the first medical facility authorizing a first action selected from the list of actions. The method can further comprise executing the first action in response to receiving the second data package. The first action can comprise a physical modification to the autonomous vehicle.

The above summary is not intended to illustrate each embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates a block diagram of an example Internet of Things (IoT) environment, in accordance with some embodiments of the present disclosure.

FIG. 2 illustrates a flowchart of an example method for managing a medical emergency in an autonomous vehicle, in accordance with some embodiments of the present disclosure.

FIG. 3 illustrates a flowchart of an example method for detecting a medical emergency, in accordance with some embodiments of the present disclosure.

FIG. 4 illustrates a flowchart of an example method for selecting a medical facility, in accordance with some embodiments of the present disclosure.

FIG. 5 illustrates a flowchart of an example method for establishing a communication channel between a passenger and a healthcare practitioner, in accordance with some embodiments of the present disclosure.

FIG. 6 illustrates a flowchart of an example method for implementing an action, in accordance with some embodiments of the present disclosure.

FIG. 7 illustrates a block diagram of an example medical system in accordance with some embodiments of the present disclosure.

FIG. 8 depicts a cloud computing environment according to some embodiments of the present disclosure.

FIG. 9 depicts abstraction model layers according to some embodiments of the present disclosure.

While the present disclosure is amenable to various modifications and alternative forms, specifics thereof have been shown by way of example in the drawings and will be described in detail. It should be understood, however, that the intention is not to limit the present disclosure to the particular embodiments described. On the contrary, the intention is to cover all modifications, equivalents, and alternatives falling within the spirit and scope of the present disclosure.

### DETAILED DESCRIPTION

Aspects of the present disclosure are directed toward automated medical assistance, and, more specifically, to managing an autonomous vehicle by a medical facility while a passenger in the autonomous vehicle is experiencing a medical emergency. While the present disclosure is not necessarily limited to such applications, various aspects of the present disclosure may be appreciated through a discussion of various examples using this context.

In some embodiments of the present disclosure, a medical system in an autonomous vehicle detects a medical condition associated with a passenger in the autonomous vehicle and, in response, sends a first data package with the medical condition, passenger, information, a location of the autonomous vehicle, and a list of actions executable by the autonomous vehicle to a selected medical facility. In some embodiments, the selected medical facility can be selected based on a location of the medical facility, a set of medical capabilities associated with the medical facility, and an availability of the medical facility. In some embodiments, a communication channel is established between the medical facility and the passenger (e.g., an audio or audio/video interaction between a healthcare practitioner associated with the medical facility and the passenger associated with the medical condition). In some embodiments, the autonomous vehicle receives a second data package from the medical facility containing instructions to manage and/or mitigate the medical condition. The autonomous vehicle can execute the instructions received in the second data package. The second data package can include instructions for routing the autonomous vehicle to the selected medical facility, routing the autonomous vehicle to a rendezvous point, or routing the autonomous vehicle to a safe location to await further assistance. The second data package can further include instructions for vehicle modifications such as, but not limited to, environmental controls (e.g., temperature, humidity, window controls, lights, sounds, etc.), emergency controls (e.g., sirens, exterior hazard lights, broadcast announcements, etc.), notification controls (e.g., presenting an estimated time of arrival on a user interface in the autonomous vehicle, presenting self-care instructions on the user interface in the autonomous vehicle, etc.), and so on.

Aspects of the present disclosure realize numerous advantages. For example, aspects of the present disclosure increase passenger safety in autonomous vehicles by communicating with a medical facility regarding a medical emergency, implementing countermeasures to increase the passenger's safety during the medical emergency, and rerouting the autonomous vehicle to the medical facility. Thus, aspects of the present disclosure increase passenger safety by automating and accelerating access to medical care for passengers experiencing a medical emergency.

Furthermore, aspects of the present disclosure can improve medical facility selection based on capabilities, location, and availability rather than exclusively based on medical facility location. Thus, aspects of the present disclosure can select a medical facility within a reasonable distance and having available and adequate resources to treat the medical emergency, thereby reducing the time until the passenger receives appropriate medical treatment. In some embodiments, aspects of the present disclosure increase processing speed of the selection process based on the procedures used to select a medical facility.

Furthermore, aspects of the present disclosure can increase the accuracy of the passenger's medical record by updating the passenger's medical record with identified medical conditions, interactions with a medical facility, and/or actions executed by the autonomous vehicle. Further still, aspects of the present disclosure increase the utility of the provided medical care by accessing a passenger's medical record and selecting appropriate medical care based on information in the passenger's medical record.

Furthermore, aspects of the present disclosure can improve redundancy and reliability by storing a medical directory within the autonomous vehicle. Thus, even in situations where communication networks may be compromised (e.g., disrupted, intermittent, unavailable, etc.), aspects of the present disclosure can nonetheless expeditiously transfer a passenger experiencing a medical emergency to an appropriate medical facility.

The aforementioned advantages are example advantages, and aspects of the present disclosure exist containing all, some, or none of the aforementioned advantages while remaining within the spirit and scope of the present disclosure.

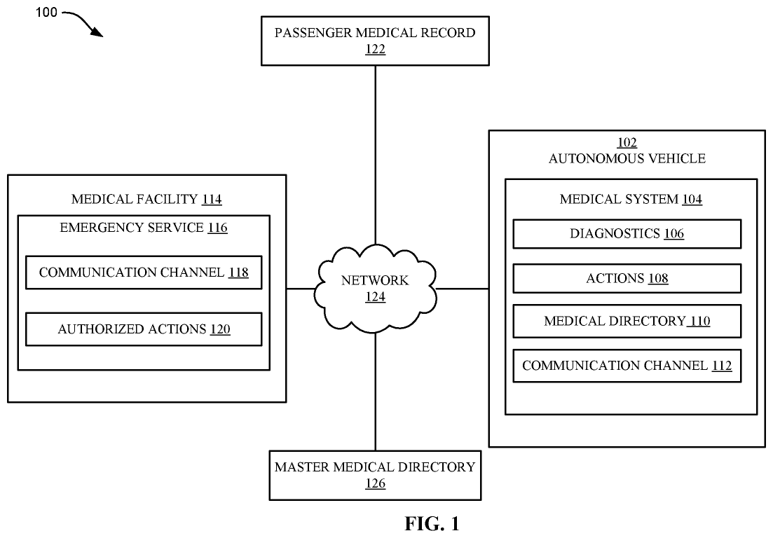

Referring now to FIG. 1, illustrated is a block diagram of an example Internet of Things (IoT) environment 100, in accordance with some embodiments of the present disclosure. IoT environment 100 can include numerous components communicatively coupled by a network 124 such as, but is not limited to, an autonomous vehicle 102, a medical facility 114, a passenger medical record 122, and a master medical directory 126.

Although the autonomous vehicle 102, the medical facility 114, the passenger medical record 122, and the master medical directory 126 are illustrated separately, in some embodiments multiple components can be integrated within one another. For example, passenger medical record 122 can be stored in (or managed by) medical facility 114. As another example, emergency service 116 (shown as part of medical facility 114) can, in some embodiments, comprise a standalone service communicatively coupled to autonomous vehicle 102, passenger medical record 122, medical facility 114, and master medical directory 126 by network 124. As another example, emergency service 116 can, in some embodiments, be integrated within autonomous vehicle 102.

Autonomous vehicle 102 can comprise a car, sedan, van, truck, sport-utility-vehicle (SUV), taxi, aerial vehicle, or a different vehicle having autonomous (or semi-autonomous) and/or automated self-driving capabilities. Autonomous vehicle 102 can have characteristics consistent with any of the autonomous levels 1-5 as defined by the Society of Automotive Engineers (SAE). Autonomous vehicle 102 can comprise medical system 104 for detecting and managing medical events. Medical system 104 can include diagnostics 106, actions 108, a medical directory 110, and a communication channel 112.

Diagnostics 106 can include sensors capable of identifying a medical condition associated with a passenger in autonomous vehicle 102. Sensors can include, but are not limited to, cameras, microphones, biometric sensors, gyroscopes, accelerometers, thermometers, and other sensors useful for collecting data associated with passenger gestures, postures, behaviors, language (e.g., words, phrases, sounds), physiology (e.g., body temperature, pulse, etc.), and environment (e.g., temperature, speed, orientation, etc.).

In some embodiments, diagnostics 106 collect at least visual information (e.g., postures, gestures, behaviors, breathing characteristics, blinking characteristics, sweating characteristics, etc.) from a camera, audio information (e.g., sounds, noises, words, phrases, tones associated with words, speeds associated with phrases, volumes associated with sounds, etc.) from a microphone, and biometric data (e.g., body temperature, heart rate, blood pressure, blood sugar level, etc.) from one or more biometric sensors.

Actions 108 can comprise actions executable by the autonomous vehicle 102. Actions 108 can include, but are not limited to, routing instructions for driving the autonomous vehicle 102 to medical facility 114, a rendezvous pint, or a safe location. Actions 108 can further include providing instructions/notifications to the passenger (e.g., informing the passenger where a rerouted vehicle is going, providing an estimated time until medical help is available, providing self-care instructions, and so on). Actions 108 can further include vehicle modifications such as, but not limited to, turning on sirens, hazard lights, changing the position of windows, changing the vehicle cabin temperature, and so on.

Although not explicitly shown, autonomous vehicle 102 further comprises the equipment necessary to execute actions 108. For example, in some embodiments, autonomous vehicle 102 can include a system configured to enable navigation such as, but not limited to, a global positioning system (GPS) or other satellite-based radionavigation system such as Differential GPS (DGPS), Galileo, GLONASS, BeiDou, GNSS augmentation, and so on. In some embodiments, autonomous vehicle 102 can include an inertial navigation system (INS) with associated components such as motion sensors (e.g., accelerometers), rotation sensors (e.g., gyroscopes), magnetic sensors (e.g., magnetometers), and a computer capable of ingesting the sensor information and calculating position, orientation, and/or velocity of the autonomous vehicle 102 based on the ingested information.

Furthermore, in some embodiments, autonomous vehicle 102 can include equipment and/or software useful for self-driving capabilities such as, but not limited to, stereo vision sensors, lidar sensors, and/or real-time locating system (RTLS) beacon systems which can be used to enable autonomous driving using algorithms such as, but not limited to, simultaneous localization and mapping (SLAM) algorithms and/or detection and tracking of other moving objects (DATMO) algorithms.

Medical directory 110 can comprise a listing of locations, capabilities, and availabilities of a plurality of healthcare facilities (e.g., hospitals, clinics, ambulances, etc.) such as, but not limited to, medical facility 114. Locations can comprise geographic locations of the medical facilities. In some embodiments, the geographic locations are dynamic, temporary, or transient locations (e.g., ambulances, medical helicopters, mobile clinics, etc.). In some embodiments, the geographic locations are static, stationary, or permanent locations (e.g., buildings).

Capabilities can comprise a listing of respective medical conditions that respective medical facilities are equipped, trained, and/or qualified to treat, manage, mitigate, or otherwise resolve. In some embodiments, capabilities are defined according to the American Trauma Society (ATS) trauma center classification (e.g., Level I, II, III, IV, or V trauma centers).

Availabilities of the medical facilities can be associated with wait times, bed availability, nurse availability, doctor availability, surgeon availability, blood type availability, medication availability, and/or other availabilities. In embodiments involving mobile medical facilities (e.g., ambulances, medical helicopters, etc.) availabilities can be associated with whether the mobile medical facility is currently responding to another call, a remaining amount of fuel (e.g., range limitations), whether the mobile medical facility is currently staffed (e.g., if a volunteer staff needs to be notified, congregated, and deployed), and so on.

Medical directory 110 can be updated at selected time intervals by communicating with master medical directory 126 via network 124. Master medical directory 126 can include updated locations (e.g., ambulance locations), capabilities, and/or availabilities.

Communication channel 112 can comprise an interaction between medical facility 114 and a passenger in autonomous vehicle 102. Communication channel 112 can comprise a textual, audio, and/or visual communication channel. Communication channel 112 can be presented using a user interface (also referred to as a user display), speakers, and/or a different component of autonomous vehicle 102. Communication channel 112 can interact with communication channel 118 of medical facility 114 via network 124.

Medical facility 114 can comprise a hospital, clinic, ambulance, emergency helicopter, emergency vehicle (e.g., fire truck, police car, mobile clinic, etc.), or a different medical facility capable of managing, treating, or otherwise mitigating a medical event associated with a passenger in autonomous vehicle 102. Medical facility 114 can update master medical directory 126 with updated locations, capabilities, and/or availabilities at respective time intervals.

Medical facility 114 can comprise emergency service 116. Emergency service 116 can be, but is not limited to, a dispatch service (e.g., a regional emergency dispatch service), a subscription-based emergency service, or a different service. Emergency service 116 comprises communication channel 118 and authorized actions 120. Communication channel 118 can interface with communication channel 112 of the autonomous vehicle 102 via network 124 to facilitate textual, verbal, and/or visual communication between a medical practitioner (e.g., doctor, nurse, dispatcher, etc.) associated with the medical facility 114 and a passenger in autonomous vehicle 102.

Authorized actions 120 can comprise actions such as, but not limited to, requesting information (e.g., a location of autonomous vehicle 102, passenger information, passenger identification, additional diagnostics 106, etc.), providing routing instructions to autonomous vehicle 102 (e.g., routing to medical facility 114, to a rendezvous point, or to a different location), providing vehicle modification instructions to the autonomous vehicle 102 (e.g., opening a window, changing a temperature in the vehicle, engaging sirens and/or hazard lights), providing passenger notifications to autonomous vehicle 102 (e.g., self-care instructions, an estimated time of arrival at medical facility 114, etc.), requesting bystander help (e.g., making an announcement over a vehicle's external speaker system requesting assistance), and so on. Self-care instructions can include, but are not limited to, medical procedures that can be performed by the passenger experiencing the medical emergency to manage the medical emergency and/or medical procedures that can be performed by a different passenger in the autonomous vehicle 102 to care for the passenger experiencing the medical emergency.

Authorized actions 120 can include automated actions (e.g., pre-authorized actions automatically and immediately implemented) and actions requiring authorization by a medical practitioner (e.g., an action requiring user input authorizing implementation of the action). Automated actions can include low risk actions (e.g., actions with limited potential side-effects) such as, but not limited to, changing an interior temperature of the vehicle (e.g., increasing temperature in a vehicle containing a passenger experiencing shock), providing notifications to the passenger, and so on. Actions requiring authorization can include altering the vehicle driving characteristics (e.g., exceeding a speed limit, driving on a shoulder of a road, etc.) that may present adverse risks to the passenger and/or the public.

Passenger medical record 122 can comprise an electronic medical record (EMR) or an electronic health record (EHR). Passenger medical record 122 can comprise demographic data, medical history data, medications, allergies, immunizations, laboratory test results, radiographic images, vital characteristics, physiological characteristics, and/or billing information. Passenger medical record 122 can be updated by autonomous vehicle 102 and/or emergency service 116 to include any one or any combination of diagnostics 106 collected by autonomous vehicle 102, authorized actions 120 authorized by medical facility 114 and implemented by autonomous vehicle 102, and/or interactions between the passenger and the medical facility 114 transmitted using communication channel 112 and communication channel 118.

Master medical directory 126 can include updated information regarding medical facility locations, capabilities, and availabilities. Master medical directory 126 can store regional, state-wide, national, and/or international medical facility information. Master medical directory 126 can be continuously updated based on input from respective medical facilities.

Network 124 can comprise a physical network, a wireless network, or a combination of physical and wireless networks. In some embodiments, network 124 comprises a cellular (e.g., 3G, 4G, 5G, etc.) network. In some embodiments, wireless networks can be realized using satellite communication, microwaves, a wireless communication protocol based on Institute of Electrical and Electronics Engineers (IEEE) standard 802.11, or a different wireless network technique and/or technology.

FIG. 1 is intended to represent the major components of an example IoT environment 100 according to embodiments of the present disclosure. In some embodiments, however, individual components can have greater or lesser complexity than shown in FIG. 1, and components other than, or in addition to those shown in FIG. 1 can be present. Furthermore, in some embodiments, various components illustrated in FIG. 1 can have greater, lesser, or different functionality than shown in FIG. 1.

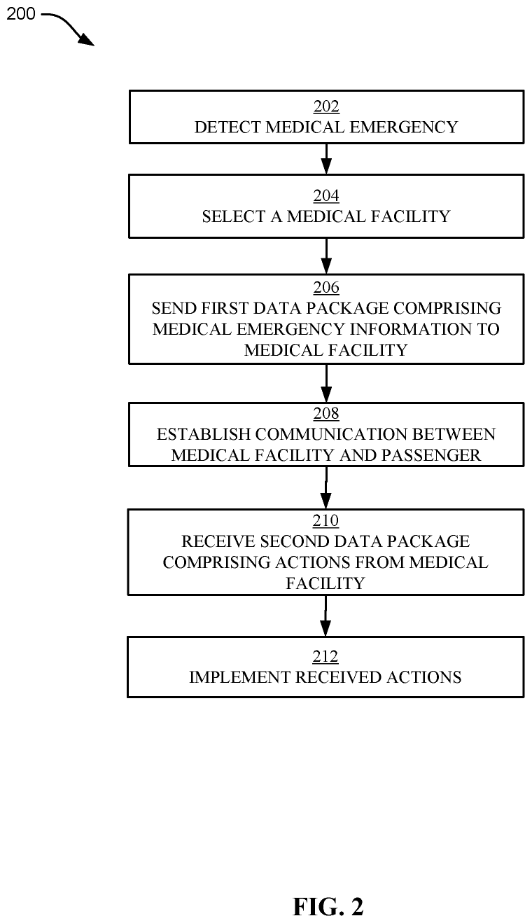

Referring now to FIG. 2, illustrated is a flowchart of an example method for managing a medical emergency in an autonomous vehicle in accordance with some embodiments of the present disclosure. The method 200 can be implemented by an autonomous vehicle (e.g., autonomous vehicle 102 of FIG. 1), a processor reading and executing instructions, or a different hardware configuration. For clarity, the method 200 will be described as being performed by an autonomous vehicle, however, the method 200 can likewise be executed by alternative configurations of hardware.

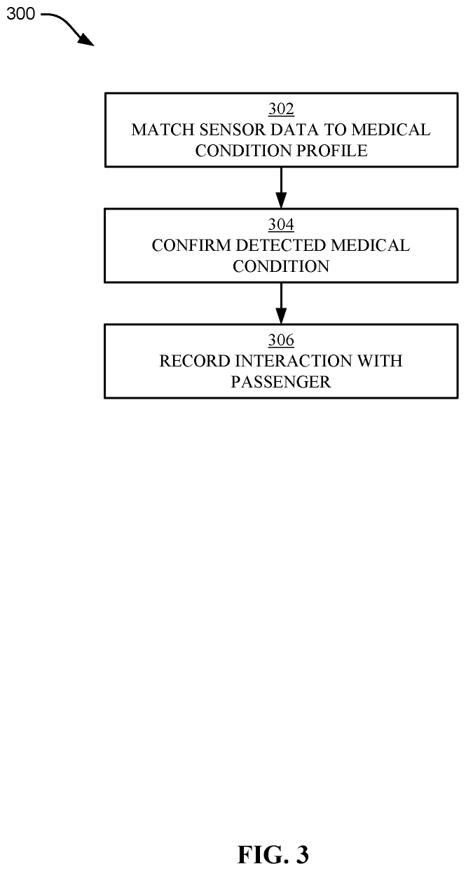

In operation 202, the autonomous vehicle can detect a medical emergency. The autonomous vehicle can detect a medical emergency using any number of techniques including, but not limited to, matching a profile of sensor data to a medical condition profile, receiving direct input from a passenger indicating a medical emergency (e.g., a passenger saying “help me”), or a different technique. In some embodiments, the autonomous vehicle detects a medical emergency based on at least visual data from a camera, audio data from a microphone, and biometric data from one or more biometric sensors. Operation 202 is discussed in more detail hereinafter with respect to FIG. 3.

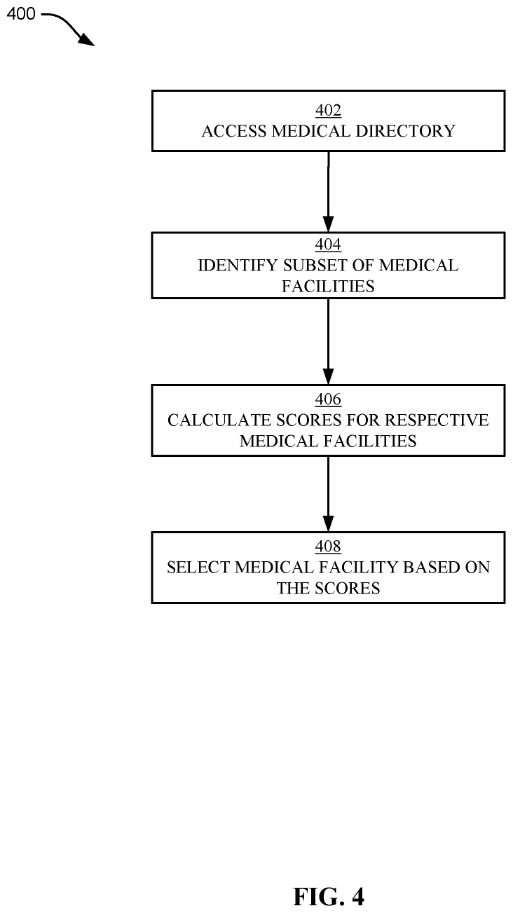

In operation 204, the autonomous vehicle can select a first medical facility using a medical facility directory (e.g., medical directory 110 of FIG. 1). The autonomous vehicle can select the first medical facility based on a location of the first medical facility, a set of medical capabilities associated with the first medical facility, and an availability of the first medical facility. Although not explicitly shown, in some embodiments, operation 204 further comprises updating (e.g., synchronizing) the medical directory stored in the autonomous vehicle with a master medical directory (e.g., master medical directory 126 of FIG. 1) before selecting a first medical facility, thereby ensuring the selection process uses the most updated information available. Operation 204 is discussed in more detail hereinafter with respect to FIG. 4.

In operation 206, the autonomous vehicle can send a first data package to the first medical facility comprising information related to the detected medical emergency. The first data package can include at least a location of the autonomous vehicle (e.g., based on GPS, INS, or a different system), information regarding the detected medical condition (e.g., sensor data, a medical condition profile), a list of actions executable by the autonomous vehicle (e.g., driving controls, audio controls, display controls, temperature controls, window controls, light controls, etc.), and/or passenger information (e.g., a passenger medical record, a passenger name, a passenger age, a passenger emergency contact, etc.). In some embodiments, the information is sent via a cellular (e.g., 3G, 4G, 5G) network.

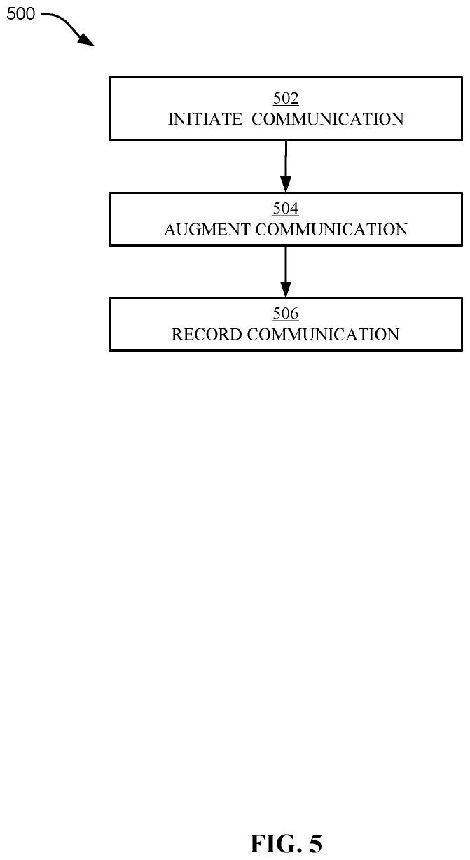

In operation 208, the autonomous vehicle can establish communication between the medical facility and a passenger in the vehicle. The communication can comprise textual, audio, video, or a combination of the aforementioned. In some embodiments, the autonomous vehicle establishes communication using a cellular network. Operation 208 is described in more detail hereinafter with respect to FIG. 5.

In operation 210, the autonomous vehicle can receive a second data package from the first medical facility (e.g., via a cellular network). The second data package can comprise instructions implementable by the autonomous vehicle (e.g., instructions for implementing one or more actions from the list of actions executable by the autonomous vehicle) such as, but not limited to, routing instructions, notification instructions, environmental control instructions, and/or other instructions.

In operation 212, the autonomous vehicle can implement the received instructions. In various embodiments, the autonomous vehicle can change a route of the autonomous vehicle according to the received instructions, change driving behavior of the autonomous vehicle according to the received instructions (e.g., exceed a speed limit, drive on a shoulder of the road, etc.), provide notifications to the passenger according to the received instructions (e.g., an estimated time of arrival, self-care instructions, etc.), modify the environmental controls of the autonomous vehicle according to the received instructions (e.g., temperature, humidity, lights, window position, noise level, etc.), and/or modify the exterior of the autonomous vehicle according to the received instructions (e.g., sirens, lights, etc.).

FIG. 2 is intended to represent the major operations of an example method for managing a medical emergency in an autonomous vehicle according to embodiments of the present disclosure. In some embodiments, however, individual operations can have greater or lesser complexity than shown in FIG. 2, and operations other than, or in addition to those shown in FIG. 2 can be present. Furthermore, in some embodiments, various operations illustrated in FIG. 2 can have greater, lesser, or different functionality than shown in FIG. 2.

Referring now to FIG. 3, illustrated is a flowchart of an example method for detecting a medical condition in accordance with some embodiments of the present disclosure. In some embodiments, the method 300 is a sub-method of operation 202 of FIG. 2. The method 300 can be implemented by an autonomous vehicle (e.g., autonomous vehicle 102 of FIG. 1), a processor reading and executing instructions, or a different hardware configuration. For clarity, the method 300 will be described as being performed by an autonomous vehicle, however, the method 300 can likewise be executed by alternative configurations of hardware.

In operation 302, the autonomous vehicle can match a set of collected sensor data to a medical condition profile stored in a medical database. The set of collected sensor data can fully match the medical condition profile, partially match the medical condition profile, or exhibit similarities to the medical condition profile despite not matching the medical condition profile. The set of collected sensor data can include visual data (e.g., from a camera), audio data (e.g., from a microphone), biometric data (e.g., from a thermometer, heart rate monitor, blood pressure monitor, blood sugar monitor, etc.), and/or environmental data (e.g., from an accelerometer, gyroscope, air composition sensor, thermometer, etc.).

In operation 304, the autonomous vehicle can confirm (e.g., verify, substantiate, check) the detected medical condition. In some embodiments, the autonomous vehicle can confirm the detected medical condition by collecting a second set of sensor data and confirming the second set of sensor data matches the medical condition profile identified in operation 302. In some embodiments, operation 304 comprises interacting with the user and requesting the user confirm the detected medical condition. For example, the autonomous vehicle can verbally or textually ask the passenger if the passenger is experiencing the detected medical condition. In another example, the autonomous vehicle can provide an indication that the autonomous vehicle will contact an emergency service in a certain amount of time (e.g., five seconds) unless receiving an input from the passenger (e.g., the passenger touching a button on a touchscreen, the passenger stating that s/he does not need medical assistance, etc.). Thus, operation 304 can reduce false positives and increase accuracy by confirming the medical condition detected in operation 302.

In operation 306, the autonomous vehicle can store a recording of the data collected during the method 300. The recorded data can include collected sensor data (e.g., a first set of sensor data and a second set of sensor data), the medical condition profile, and/or any input from the passenger confirming the medical condition, denying the medical condition, or failing to respond to an interaction regarding the medical condition. In some embodiments, the recording of the data can be automatically stored in a passenger medical record (e.g., passenger medical record 122 of FIG. 1).

FIG. 3 is intended to represent the major operations of an example method for detecting a medical condition in an autonomous vehicle according to embodiments of the present disclosure. In some embodiments, however, individual operations can have greater or lesser complexity than shown in FIG. 3, and operations other than, or in addition to those shown in FIG. 3 can be present. Furthermore, in some embodiments, various operations illustrated in FIG. 3 can have greater, lesser, or different functionality than shown in FIG. 3.

Referring now to FIG. 4, illustrated is a flowchart of an example method for selecting a medical facility in accordance with some embodiments of the present disclosure. In some embodiments, the method 400 is a sub-method of operation 204 of FIG. 2. The method 400 can be implemented by an autonomous vehicle (e.g., autonomous vehicle 102 of FIG. 1), a processor reading and executing instructions, or a different hardware configuration. For clarity, the method 400 will be described as being performed by an autonomous vehicle, however, the method 400 can likewise be executed by alternative configurations of hardware.

In operation 402, the autonomous vehicle can access a medical directory (e.g., medical directory 110 of FIG. 1). The medical directory can comprise a database storing a plurality of medical facilities (e.g., hospitals, clinics, ambulances, emergency helicopters, other emergency vehicles, etc.). The medical directory can indicate, for each of the plurality of medical facilities, a location of the medical facility, a set of medical conditions treatable at the medical facility, and an availability of the medical facility.

In operation 404, the autonomous vehicle can identify a subset of medical facilities. In some embodiments, the subset of medical facilities can comprise at least the medical facilities associated with a set of capabilities matching the medical condition of the passenger and/or medical facilities within a certain radius of the autonomous vehicle (e.g., a 100-mile radius, a 30-mile radius, a 15-minute travel time radius, etc.). Thus, operation 404 can accelerate selection of a medical facility by first reducing the total number of medical facilities (e.g., a national or international registry of medical facilities) to a subset of medical facilities based on capabilities and/or locations in light of the location of the autonomous vehicle and the medical condition of the passenger in the autonomous vehicle.

In operation 406, the autonomous vehicle can calculate respective scores for respective medical facilities in the subset of medical facilities. The respective scores can be based on capabilities, locations, and availabilities associated with the subset of medical facilities. Capabilities, locations, and availabilities can be weighted, combined, and otherwise manipulated in one or more functions to generate a score. In some embodiments, operation 406 first updates the information of the subset of medical facilities by retrieving updated information from a master medical directory (e.g., master medical directory 126 of FIG. 1), thereby increasing accuracy of the calculated scores and/or reducing the processing time associated with the updating by only updating records associated with the subset of medical facilities.

In some embodiments, the scores calculated in operation 406 indicate a total estimated time until a passenger receives appropriate medical care for each medical facility in the subset of medical facilities. In such embodiments, the scores account for travel time (e.g., accounting for distance, traffic, speed, etc.) and wait time (e.g., accounting for availabilities of needed personnel associated with each medical facility). In such embodiments, lower scores may indicate shorter wait times until appropriate medical care is received.

In operation 408, the autonomous vehicle can select a medical facility based on the scores calculated in operation 406. In some embodiments, operation 408 selects the medical facility having a best score (e.g., a lowest score or highest score depending on the scoring mechanism) or best rank.

FIG. 4 is intended to represent the major operations of an example method for selecting a medical facility according to embodiments of the present disclosure. In some embodiments, however, individual operations can have greater or lesser complexity than shown in FIG. 4, and operations other than, or in addition to those shown in FIG. 4 can be present. Furthermore, in some embodiments, various operations illustrated in FIG. 4 can have greater, lesser, or different functionality than shown in FIG. 4.

Referring now to FIG. 5, illustrated is a flowchart of an example method for initiating a communication channel between a healthcare practitioner and a passenger in accordance with some embodiments of the present disclosure. In some embodiments, the method 500 is a sub-method of operation 208 of FIG. 2. The method 500 can be implemented by an autonomous vehicle (e.g., autonomous vehicle 102 of FIG. 1), a processor reading and executing instructions, or a different hardware configuration. For clarity, the method 500 will be described as being performed by an autonomous vehicle, however, the method 500 can likewise be executed by alternative configurations of hardware.

In operation 502, the autonomous vehicle initiates a textual, audio, and/or video communication channel between a healthcare practitioner associated with a medical facility and a passenger in the autonomous vehicle. In some embodiments, the communication channel utilizes a user interface and/or a speaker system in the autonomous vehicle.

In operation 504, the autonomous vehicle augments the textual, audio, and/or video communication. For example, the autonomous vehicle can use voice-to-text software to present textual subtitles on a user interface in the autonomous vehicle containing the spoken words from the healthcare practitioner and/or the passenger. In some embodiments, the autonomous vehicle provides translation services between a healthcare practitioner speaking a first language and a passenger speaking a second language. In such embodiments, the autonomous vehicle can implement instructions for converting words spoken in the first language to text presented in the second language and vice versa.

In operation 506, the autonomous vehicle can record the communication and store the communication in the medical record of the passenger (e.g., passenger medical record 122).

FIG. 5 is intended to represent the major operations of an example method for establishing a communication channel between a healthcare practitioner and a passenger according to embodiments of the present disclosure. In some embodiments, however, individual operations can have greater or lesser complexity than shown in FIG. 5, and operations other than, or in addition to those shown in FIG. 5 can be present. Furthermore, in some embodiments, various operations illustrated in FIG. 5 can have greater, lesser, or different functionality than shown in FIG. 5.

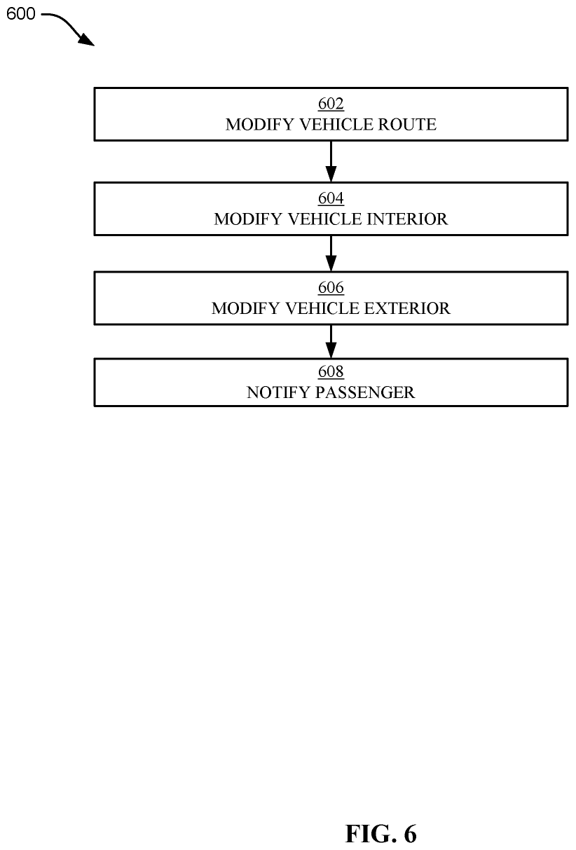

Referring now to FIG. 6, illustrated is a flowchart of an example method for implementing a vehicle action in accordance with some embodiments of the present disclosure. In some embodiments, the method 600 is a sub-method of operation 212 of FIG. 2. The method 600 can be implemented by an autonomous vehicle (e.g., autonomous vehicle 102 of FIG. 1), a processor reading and executing instructions, or a different hardware configuration. For clarity, the method 600 will be described as being performed by an autonomous vehicle, however, the method 600 can likewise be executed by alternative configurations of hardware.

In operation 602, the autonomous vehicle can modify the route according to instructions received from the healthcare facility in the second data package. Modifying the vehicle route can include, but is not limited to, stopping the vehicle in a safe location (e.g., a side of the road, a pickup point, a parking lot, etc.), driving the vehicle to a rendezvous point (e.g., to meet an ambulance, an emergency helicopter, etc.), or driving the vehicle directly to a medical facility (e.g., a hospital, a clinic, etc.). In some embodiments, the medical facility provides the autonomous vehicle with a destination and the autonomous vehicle uses internal navigation software to select a route to the destination. In other embodiments, the medical facility provides the autonomous vehicle with the destination and a route to the destination and the autonomous vehicle travels to the destination according to the provided route. In some embodiments, the route can include directions (e.g., compass readings), waypoints (e.g., grid coordinates), speeds, accelerations, decelerations, and other instructions (e.g., drive on shoulder of road to bypass traffic while traveling to the medical facility).

In operation 604, the autonomous vehicle can modify an interior environment of the autonomous vehicle according to instructions received in the second data package from the medical facility. The autonomous vehicle can modify an interior environment of the autonomous vehicle by, for example, changing a temperature, humidity, air composition, amount of noise, amount of light, or a different factor of the interior environment. In operation 604, the autonomous vehicle can change a position of one or more windows (e.g., open windows to provide fresh air to the passenger), change a temperature control and/or fan speed of the autonomous vehicle (e.g., heat up an interior of a vehicle for a passenger going into shock), change a radio control (e.g., to attempt to wake a semi-conscious or unconscious passenger), change a lighting level control, and/or change other controls.

In operation 606, the autonomous vehicle can modify the exterior of the autonomous vehicle according to instructions received in the second data package from the medical facility. The exterior of the autonomous vehicle can be modified by changing lighting (e.g., turning on hazard lights, emergency lights, rapidly turning the headlights on and off, or other lighting modifications) and/or projecting sounds outside the vehicle (e.g., sirens, announcements, etc.).

In operation 608, the autonomous vehicle can provide a notification to a passenger. The notification can include, but is not limited to, a text notification, a verbal notification, a pictographic notification, or a different notification configured to notify the passenger of the medical condition, an arrival time, a medical facility, or other relevant information. The notification can be provided to a user interface in the autonomous vehicle and/or to a device associated with the passenger (e.g., a mobile phone, a wearable device, etc.). The notification can include self-care instructions for the passenger (e.g., apply pressure to a wound, take deep breaths, etc.), an estimated arrival time (e.g., 3 minutes until arrival at the hospital), information about the medical emergency (e.g., informing the passenger he or she may be experiencing a heart attack and the autonomous vehicle is driving to a medical facility for medical treatment), or a different notification.

FIG. 6 is intended to represent the major operations of an example method for implementing an action according to embodiments of the present disclosure. In some embodiments, however, individual operations can have greater or lesser complexity than shown in FIG. 6, and operations other than, or in addition to those shown in FIG. 6 can be present. Furthermore, in some embodiments, various operations illustrated in FIG. 6 can have greater, lesser, or different functionality than shown in FIG. 6.

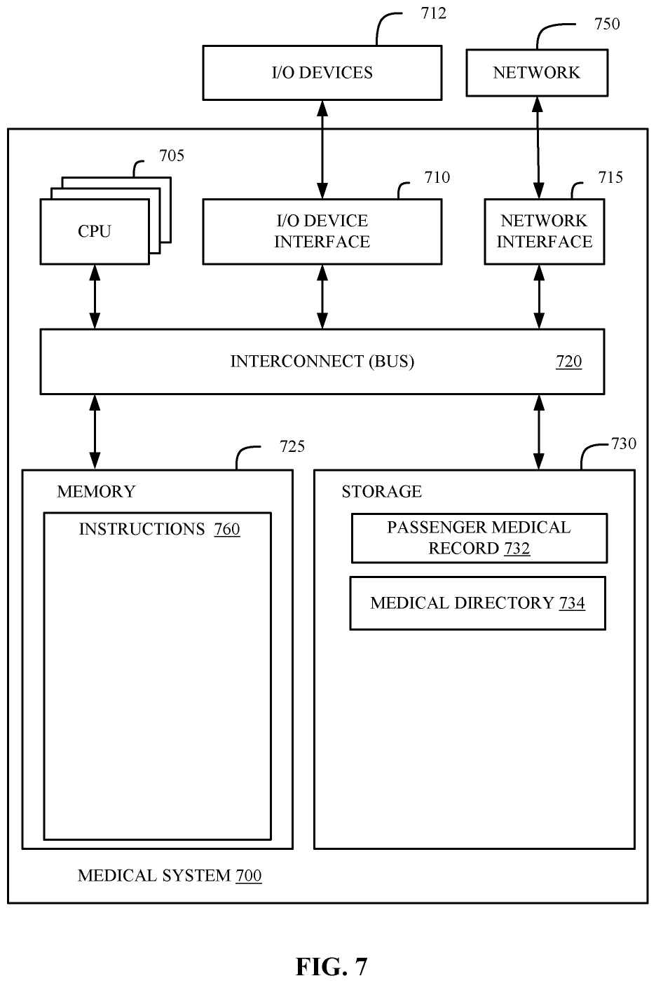

FIG. 7 illustrates a block diagram of a medical system 700 in accordance with some embodiments of the present disclosure. In some embodiments, medical system 700 is consistent with medical system 104 of FIG. 1. In various embodiments, medical system 700 performs any of the methods described in FIGS. 2-6. In some embodiments, medical system 700 provides instructions for one or more of the methods described in FIGS. 2-6 to a client machine (e.g., an autonomous vehicle) such that the client machine executes the method, or a portion of the method, based on the instructions provided by the medical system 700.

The medical system 700 includes a memory 725, storage 730, an interconnect (e.g., BUS) 720, one or more CPUs 705 (also referred to as processors 705 herein), an I/O device interface 710, I/O devices 712, and a network interface 715.

Each CPU 705 retrieves and executes programming instructions stored in the memory 725 or storage 730. The interconnect 720 is used to move data, such as programming instructions, between the CPUs 705, I/O device interface 710, storage 730, network interface 715, and memory 725. The interconnect 720 can be implemented using one or more busses. The CPUs 705 can be a single CPU, multiple CPUs, or a single CPU having multiple processing cores in various embodiments. In some embodiments, a CPU 705 can be a digital signal processor (DSP). In some embodiments, CPU 705 includes one or more 3D integrated circuits (3DICs) (e.g., 3D wafer-level packaging (3DWLP), 3D interposer based integration, 3D stacked ICs (3D-SICs), monolithic 3D ICs, 3D heterogeneous integration, 3D system in package (3DSiP), and/or package on package (PoP) CPU configurations). Memory 725 is generally included to be representative of a random access memory (e.g., static random access memory (SRAM), dynamic random access memory (DRAM), or Flash). The storage 730 is generally included to be representative of a non-volatile memory, such as a hard disk drive, solid state device (SSD), removable memory cards, optical storage, or flash memory devices. In an alternative embodiment, the storage 730 can be replaced by storage area-network (SAN) devices, the cloud, or other devices connected to the medical system 700 via the I/O devices interface 710 or a network 750 via the network interface 715.

In some embodiments, the memory 725 stores instructions 760 and the storage 730 stores passenger medical record 732 and medical directory 734. However, in various embodiments, the instructions 760, the passenger medical record 732, and the medical directory 734 are stored partially in memory 725 and partially in storage 730, or they are stored entirely in memory 725 or entirely in storage 730, or they are accessed over a network 750 via the network interface 715.

Passenger medical record 732 can comprise an electronic medical record (also referred to as an electronic health record) of a passenger. In some embodiments, passenger medical record 732 is consistent with passenger medical record 122 of FIG. 1. Passenger medical record 732 can include demographics, medical histories, medications, conditions, allergies, vital characteristics, physiological information, laboratory test results, and/or other medical-related information. Aspects of the present disclosure amend passenger medical record 732 to include a recorded communication channel, a first set of diagnostic sensor data, a second set of diagnostic sensor data, a medical condition profile, and/or a recorded interaction with a passenger.

Medical directory 734 can comprise a database listing locations, capabilities, and/or availabilities of medical facilities (e.g., regionally, state-wide, nationally, internationally, etc.). In some embodiments, medical directory 734 is consistent with medical directory 110 and/or master medical directory 126 of FIG. 1. Aspects of the present disclosure use medical directory 734 to select a medical facility based on location, capability, and availability of the medical facility. In some embodiments, the medical system 700 uses medical directory 734 to identify a medical facility capable of resolving a passenger's medical condition in the shortest amount of time.

The instructions 760 are processor executable instructions for executing any portion of, any combination of, or all of the methods previously discussed in FIGS. 2-6.

In various embodiments, the I/O devices 712 include an interface capable of presenting information and receiving input. For example, I/O devices 712 can present information to a passenger (or healthcare practitioner) interacting with medical system 700 and receive input from the passenger (or healthcare practitioner).

Medical system 700 is connected to the network 750 via the network interface 715. In some embodiments, network 750 is consistent with network 124 of FIG. 1.

FIG. 7 is intended to represent the major components of an example medical system 700 according to embodiments of the present disclosure. In some embodiments, however, individual components can have greater or lesser complexity than shown in FIG. 7, and components other than, or in addition to those shown in FIG. 7 can be present. Furthermore, in some embodiments, various components illustrated in FIG. 7 can have greater, lesser, or different functionality than shown in FIG. 7.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

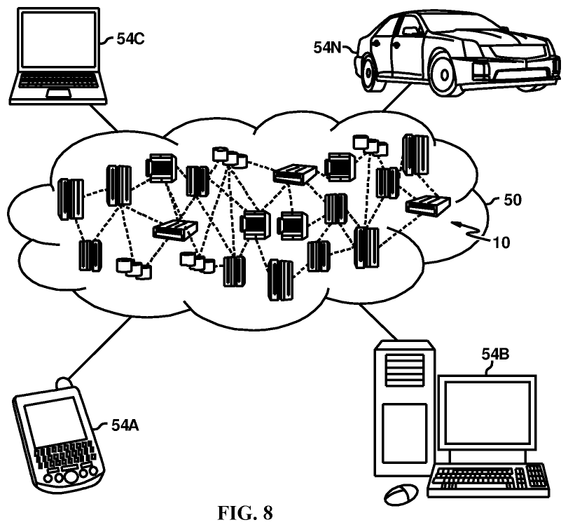

Referring now to FIG. 8, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 8 are intended to be illustrative only and that computing nodes 10 and cloud computing environment 50 can communicate with any type of computerized device over any type of network and/or network addressable connection (e.g., using a web browser).

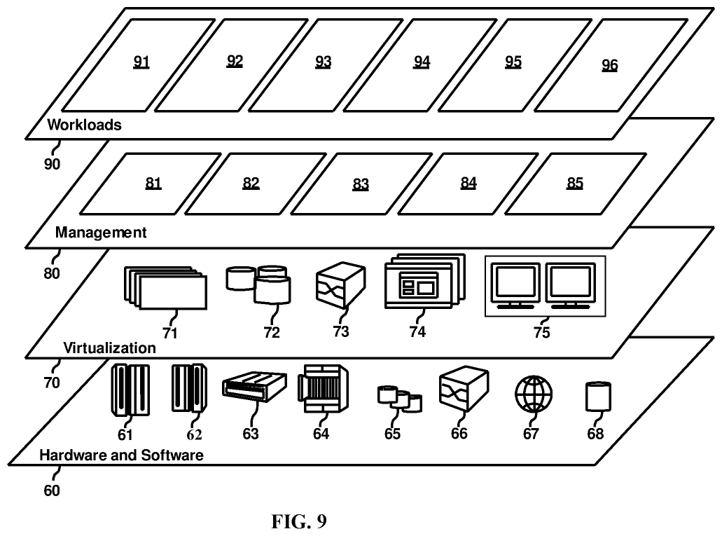

Referring now to FIG. 9, a set of functional abstraction layers provided by cloud computing environment 50 (FIG. 8) is shown. It should be understood in advance that the components, layers, and functions shown in FIG. 9 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and virtual medical assistance 96.

Embodiments of the present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or subset of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While it is understood that the process software (e.g., any of the instructions stored in instructions 760 of FIG. 7 and/or any software configured to perform any subset of the methods described with respect to FIGS. 2-6) may be deployed by manually loading it directly in the client, server, and proxy computers via loading a storage medium such as a CD, DVD, etc., the process software may also be automatically or semi-automatically deployed into a computer system by sending the process software to a central server or a group of central servers. The process software is then downloaded into the client computers that will execute the process software. Alternatively, the process software is sent directly to the client system via e-mail. The process software is then either detached to a directory or loaded into a directory by executing a set of program instructions that detaches the process software into a directory. Another alternative is to send the process software directly to a directory on the client computer hard drive. When there are proxy servers, the process will select the proxy server code, determine on which computers to place the proxy servers' code, transmit the proxy server code, and then install the proxy server code on the proxy computer. The process software will be transmitted to the proxy server, and then it will be stored on the proxy server.

Embodiments of the present invention may also be delivered as part of a service engagement with a client corporation, nonprofit organization, government entity, internal organizational structure, or the like. These embodiments may include configuring a computer system to perform, and deploying software, hardware, and web services that implement, some or all of the methods described herein. These embodiments may also include analyzing the client's operations, creating recommendations responsive to the analysis, building systems that implement subsets of the recommendations, integrating the systems into existing processes and infrastructure, metering use of the systems, allocating expenses to users of the systems, and billing, invoicing (e.g., generating an invoice), or otherwise receiving payment for use of the systems.

What is claimed is:

1. A computer-implemented method for managing an autonomous vehicle during a medical emergency, the method comprising:
detecting, based on sensor data received from a plurality of sensors communicatively coupled to the autonomous vehicle, a medical condition associated with a passenger in the autonomous vehicle;
identifying, in response to detecting the medical condition and based on information in a medical facility directory stored in a computer-readable storage medium in the autonomous vehicle, a first medical facility from a set of medical facilities based on a location of the autonomous vehicle, a location of the first medical facility, the medical condition, a set of medical capabilities associated with the first medical facility, and an availability of the first medical facility;
sending, in response to identifying the first medical facility, a first data package to the first medical facility using a cellular network, wherein the first data package comprises the medical condition, passenger information, the location of the autonomous vehicle, and a list of actions executable by the autonomous vehicle;
receiving, from the cellular network and in response to sending the first data package, a second data package from the first medical facility authorizing a first action selected from the list of actions; and
executing the first action in response to receiving the second data package, wherein the first action comprises a physical modification to the autonomous vehicle.

2. The method according to claim 1, further comprising:
providing, in response to identifying the first medical facility, a communication channel between the passenger and a health care practitioner associated with the first medical facility, wherein the communication channel comprises a visual component and an audio component.

3. The method according to claim 2, wherein providing a communication channel further comprises:
initiating the communication channel including a visual component presented on a user display in the autonomous vehicle and an audio component using a speaker system of the autonomous vehicle;
augmenting the communication channel by presenting text of verbal communication on the user display; and
storing a recording of the communication channel in a medical record associated with the passenger.

4. The method according to claim 3, wherein augmenting the communication channel further comprises receiving audio data spoken in a first language and converting the audio data to textual data presented on the user display in a second language.

5. The method according to claim 1, wherein detecting a medical emergency further comprises:
matching a first set of sensor data to a first medical condition profile, wherein the first set of sensor data comprises video data, audio data, and biometric data;
confirming the first medical condition profile by collecting a second set of sensor data comprising video data, audio data, and biometric data;
verifying the second set of sensor data matches the first medical condition profile; and
recording the first set of sensor data, the second set of sensor data, and the first medical condition profile in a medical record associated with the passenger.

6. The method according to claim 1, wherein identifying a first medical facility further comprises:
accessing the medical facility directory, wherein the medical facility directory comprises a plurality of medical facilities, wherein the plurality of medical facilities comprises hospitals and clinics;
wherein the medical facility directory associates respective medical facilities with respective capabilities, wherein respective capabilities comprise respective medical conditions treatable by respective medical facilities;
wherein the medical facility directory associates respective medical facilities with respective locations; and
wherein the medical facility directory associates respective medical facilities with respective availabilities;
identifying a subset of the medical facilities, wherein each medical facility in the subset of the medical facilities is associated with a capability matching the medical condition;
calculating respective scores for respective medical facilities in the subset of the medical facilities, wherein respective scores are based on respective locations of respective medical facilities in the subset of medical facilities, respective availabilities of respective medical facilities in the subset of medical facilities, and the location of the autonomous vehicle; and
selecting the first medical facility based on the respective scores.

7. The method according to claim 1, wherein executing the first action further comprises:
modifying a route of the autonomous vehicle to the first medical facility; and
notifying the passenger of an estimated time of arrival at the first medical facility.

8. The method according to claim 7, wherein the first action further comprises:
modifying an interior environment of the autonomous vehicle using an environmental control system in the autonomous vehicle; and
turning on hazard lights on the autonomous vehicle.

9. A system for managing an autonomous vehicle during a medical emergency, the system comprising:
a processor; and
a computer-readable storage medium storing a medical facility directory and program instructions for emergency service functionality which, when executed by the processor, are configured to cause the processor to perform a method comprising:
detecting, based on sensor data received from a plurality of sensors communicatively coupled to the autonomous vehicle, a medical condition associated with a passenger in the autonomous vehicle;
identifying, in response to detecting the medical condition and based on information in the medical facility directory, a first medical facility from a set of medical facilities based on a location of the autonomous vehicle, a location of the first medical facility, the medical condition, a set of medical capabilities associated with the first medical facility, and an availability of the first medical facility;
sending, in response to identifying the first medical facility, a first data package to the first medical facility using a cellular network, wherein the first data package comprises the medical condition, passenger information, the location of the autonomous vehicle, and a list of actions executable by the autonomous vehicle;
receiving, from the cellular network and in response to sending the first data package, a second data package from the first medical facility authorizing a first action selected from the list of actions; and
executing the first action in response to receiving the second data package, wherein the first action comprises a physical modification to the autonomous vehicle.

10. The system according to claim 9, wherein the program instructions are stored in a computer readable storage medium in the autonomous vehicle, and wherein the program instructions were downloaded over a network from a remote data processing system.

11. The system according to claim 9, wherein the program instructions are stored in a computer readable storage medium in a server data processing system, and wherein the instructions are downloaded over a network to the autonomous vehicle to provide emergency service functionality to the autonomous vehicle.

12. The system according to claim 11, wherein the program instructions are configured to cause the processor to perform a method further comprising:
metering use of the emergency service functionality in the autonomous vehicle; and
generating an invoice in response to metering use of the emergency service functionality.

13. The system according to claim 9, the program instructions further configured to cause the processor to perform a method further comprising:
providing, in response to identifying the first medical facility, a communication channel between the passenger and a health care practitioner associated with the first medical facility, wherein the communication channel comprises a visual component and an audio component, wherein providing the communication channel further comprises:
initiating the communication channel including a visual component presented on a user display in the autonomous vehicle and an audio component using a speaker system of the autonomous vehicle;
augmenting the communication channel by presenting text of verbal communication on the user display; and
storing a recording of the communication channel in a medical record associated with the passenger.

14. The system according to claim 9, wherein identifying a first medical facility further comprises:
accessing the medical facility directory, wherein the medical facility directory comprises a plurality of medical facilities, wherein the plurality of medical facilities comprises hospitals and clinics;
wherein the medical facility directory associates respective medical facilities with respective capabilities, wherein respective capabilities comprise respective medical conditions that are treatable by respective medical facilities;
wherein the medical facility directory associates respective medical facilities with respective locations; and
wherein the medical facility directory associates respective medical facilities with respective availabilities;
identifying a subset of the medical facilities, wherein each medical facility in the subset of the medical facilities is associated with a capability matching the medical condition;
calculating respective scores for respective medical facilities in the subset of the medical facilities, wherein respective scores are based on respective locations of respective medical facilities in the subset of the medical facilities, respective availabilities of respective medical facilities in the subset of the medical facilities, and the location of the autonomous vehicle; and
selecting the first medical facility based on calculating the respective scores.

15. The system according to claim 9, wherein executing the first action further comprises:
modifying a route of the autonomous vehicle to the first medical facility; and
notifying the passenger of an estimated time of arrival at the first medical facility.

16. A computer program product for managing an autonomous vehicle during a medical emergency, the computer program product comprising a computer readable storage medium, wherein the computer readable storage medium does not comprise a transitory signal per se, wherein the computer readable storage medium stores instructions executable by a processor to cause the processor to perform a method comprising:
detecting, based on sensor data received from a plurality of sensors communicatively coupled to the autonomous vehicle, a medical condition associated with a passenger in the autonomous vehicle;
identifying, in response to detecting the medical condition and based on information in a medical facility directory stored in the computer readable storage medium, a first medical facility from a set of medical facilities based on a location of the autonomous vehicle, a location of the first medical facility, the medical condition, a set of medical capabilities associated with the first medical facility, and an availability of the first medical facility;
sending, in response to identifying the first medical facility, a first data package to the first medical facility using a cellular network, wherein the first data package comprises the medical condition, passenger information, the location of the autonomous vehicle, and a list of actions executable by the autonomous vehicle;
receiving, from the cellular network and in response to sending the first data package, a second data package from the first medical facility authorizing a first action selected from the list of actions; and
executing the first action in response to receiving the second data package, wherein the first action comprises a physical modification to the autonomous vehicle.

17. The computer program product according to claim 16, wherein the instructions are further configured to cause the processor to perform a method further comprising:
providing, in response to identifying the first medical facility, a communication channel between the passenger in the autonomous vehicle and a health care practitioner associated with the first medical facility, wherein the communication channel comprises a visual component and an audio component.

18. The computer program product according to claim 17, wherein providing a communication channel further comprises:
initiating the communication channel in the autonomous vehicle by presenting a visual component on a user display in the autonomous vehicle and an audio component using a speaker system of the autonomous vehicle;
initiating the communication channel at the first medical facility by presenting a visual component on a computer at the first medical facility and an audio component using the computer at the first medical facility;
augmenting the communication channel by presenting text of verbal communication on the user display in the autonomous vehicle and on the computer at the first medical facility; and
storing a recording of the communication channel in a medical record associated with the passenger.

19. The computer program product according to claim 18, wherein augmenting the communication channel further comprises receiving audio data spoken in a first language and converting the audio data to textual data presented on the user display in a second language.

20. The computer program product according to claim 16, wherein the first action comprises a temperature modification in the autonomous vehicle, a notification presented to the passenger in the autonomous vehicle, and self-care instructions presented to the passenger in the autonomous vehicle.
